(19) United States
(12) Patent Application Publication
(10) Pub. No.: US 2012/0004124 A1
(43) Pub. Date: Jan. 5, 2012

(54) MARKERS OF REGULATORY T CELL ACTIVATION

(76) Inventors: Joachim Schultze, Koenigswinter (DE); Marc Beyer, Cologne (DE)

(21) Appl. No.: 12/972,954

(22) Filed: Dec. 20, 2010

Related U.S. Application Data
(60) Provisional application No. 61/288,790, filed on Dec. 21, 2009.

(51) Int. Cl. C40B 30/04; C12N 5/0783

(52) U.S. Cl. 506/9; 435/325

(57) ABSTRACT

Aspects of the present invention include novel marker genes for the identification, isolation, and characterization of activated suppressive and/or regulatory T cells. Use of the isolated activated suppressive and/or regulatory T cells as well as screening assays to identify agents that inhibit or activate suppressive and/or regulatory T cells are also provided.

### CROSS REFERENCE TO RELATED APPLICATION

This application claims priority from U.S. Provisional Application Ser. No. 61/288,790 filed on Dec. 21, 2009, the entirety of which is incorporated herein by reference.

### INTRODUCTION

As early as 1971, a so-called “suppressor” cell population was first described by Gershon and Kondo when they transferred antigen-specific tolerance to naïve animals by transferring antigen-experienced T cells (Gershon and Kondo, Immunology; 21: 903-914 (1971)). Due to conflicting results the concept of T cell suppression however fell into oblivion in the late 1980s.

Sakaguchi et al. were the first to describe now termed “regulatory” T cells (Treg cells) by identifying a population of CD4⁺ T cells highly expressing CD25 and preventing autoimmunity in a murine model (Sakaguchi et al., J. Immunol. 155: 1151-1164 (1995)). In the following years a number of reports enlightened major aspects of Treg cell biology, characterizing different T cell subpopulations with regulatory properties including naturally occurring CD4⁺CD25^{high} Treg cells, induced Treg cells, e.g. Tr1 and TH3 cells, as well as adaptive CD4⁺CD25^{high} Treg cells developing in the periphery by conversion of CD4⁺CD25⁻ T cells. All these different T cell populations with regulatory function coexist and contribute to immune suppression (Mills and McGuirk, Semin. Immunol.; 16: 107-117 (2004); Sakaguchi, Annu. Rev. Immunol.; 22: 531-562 (2004); Sakaguchi, Nat. Immunol.; 6: 345-352 (2005); Vigouroux et al., Blood; 104: 26-33 (2004)).

Similar to murine Treg cells, CD4⁺CD25^{high} human Treg cells have been identified (see, e.g., Jonuleit et al. Journal of Experimental Medicine (2000) vol 192, p 1213; U.S. Pat. No. 6,358,506; both of which are incorporated herein by reference) and shown to possess immunosuppressive activity. Given their immunosuppressive activity, Treg cells have become an important target for therapies to treat a number of different conditions characterized by aberrant immune responses.

### SUMMARY

Aspects of the present invention include novel marker genes for the identification, isolation, and characterization of activated suppressive and/or regulatory T cells. Use of the isolated activated suppressive and/or regulatory T cells as well as screening assays to identify agents that inhibit or activate suppressive and/or regulatory T cells are also provided.

### DEFINITIONS

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. As used herein, the term “gene” or “recombinant gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like.

As is understood in the art, a “polypeptide” is a chain of amino acids linked to one another by peptide bonds. A “protein” can be made up of one or more poly-peptides, while a “peptide” is generally understood to be (or include) a fragment of a polypeptide, and to consist of a chain of peptide bond-linked amino acids that is shorter in length than a full length polypeptide from which it may be de-rived.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, procaryotic or eukaryotic mRNA, genomic DNA sequences from viral, procaryotic or eukaryotic DNA, and even synthetic DNA sequences. A transcription termination sequence may be located 3′ to the coding sequence.

The terms “reference” and “control” are used interchangeably to refer to a known value or set of known values against which an observed value may be compared. As used herein, known means that the value represents an understood parameter, e.g., a level of expression of a marker gene in an activated or resting Treg cell.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides. The terms “protein” and “polypeptide” used in this application are interchangeable. “Polypeptide” refers to a polymer of amino acids (amino acid sequence) and does not refer to a specific length of the molecule. Thus peptides and oligopeptides are included within the definition of polypeptide. This term does also refer to or include post-translational modifications of the polypeptide, for example, glycosylations, acetylations, phosphorylation and the like. Included within the definition are, for example, polypeptides containing one or more analogs of an amino acid, polypeptides with substituted linkages, as well as other modifications known in the art, both naturally occurring and non-naturally occurring. The term “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

By “histocompatibility antigen” is meant a molecule, such as a major histocompatibility complex (MHC) class I, MHC class II, or minor histocompatibility anti-gen, that mediates interactions of cells of the immune system with each other and with other cell types. Examples of histocompatibility antigens include MHC class I antigens, such as HLA-A (e.g., A1, A2, A3, A11, A24, A31, A33, and A38), HLA-B, and HLA-C, MHC class II antigens, such as HLA-DR, HLA-DQ, HLA-DX, HLA-DO, HLA-DZ, and HLA-DP, and minor histocompatibility antigens, such as HA-1.

By “generating CTLs” is meant an in vivo, in vitro, or ex vivo process by which CTLs (e.g., CTLs specific for a protein listed in Table 1) are activated (e.g., stimulated to grow and divide) and/or selected.

A peptide is said to “specifically bind” to an MHC antigen if the peptide adheres to a histocompatibility antigen under physiological conditions. For example, such binding can be similar to that of a peptide antigen that is naturally processed and presented in the context of MHC in an antigen presenting cell.

An antibody or cytotoxic T lymphocyte (CTL) is said to “specifically recognize” a polypeptide if it binds to the polypeptide or peptide (direct binding by an antibody; binding by the antigen receptor for a CTL), but does not substantially bind to other, unrelated polypeptides or peptides.

A CTL is said to “specifically kill” a cell if it specifically recognizes and lyses a cell that expresses an antigen to which it has been activated, but does not substantially recognize or lyse cells not expressing the antigen.

A polypeptide is “presented” if it is displayed on the extracellular surface of a cell (e.g., an antigen presenting cell), such that it can result in the in vivo, ex vivo, or in vitro generation of specific CTLs

By “sample” is meant a tumor or tissue biopsy, a lymph node biopsy, bone mar-row, cells, blood, serum, urine, stool, sputum, saliva, or other specimen obtained from a patient. A sample can be analyzed to determine the level of gene expression, e.g., to identify activated Treg cells, Treg specific CTLs, or the level of any other immune response indicator (e.g., a cytokine) in the patient from whom it was taken by methods that are known in the art. For example, flow cytometry can be used to identify (and quantitate) activated Treg cells based on their protein expression, and ELISPOT can be used to measure cytokine levels.

By “Treg cell elimination” is meant any therapy (e.g., chemotherapy, radiation therapy, administration of a specific CTLs, administration of an APC presenting a peptide of or vaccination with a protein/peptide of interest (i.e., encoded by a Table 1 gene), a nucleic acid molecule encoding a protein of interest, or a fragment thereof, to enhance an anti Treg cell immune response) administered either alone or in combination with other therapies, that influences Treg cell frequencies in at least some patients to which the treatment is administered. For example, Treg cell elimination can partially or completely reduce or inhibit Treg cells. Furthermore, Treg cell elimination can be prophylactic, in that it inhibits or prevents the development of new Treg cells in healthy individuals, in patients that are in remission from cancer, have metastatic cancer, or have a high risk of developing cancer.

By “inhibiting the development of Treg cells” is meant administering a protective therapy to a subject adjudged to have a higher than average risk of developing high frequencies of activated Treg cells.

By “pharmaceutically acceptable carrier” is meant a carrier that is physiologically acceptable to a patient, while retaining the therapeutic properties of the com-pound with which it is administered. One exemplary pharmaceutically acceptable carrier is physiological saline. Other physiologically acceptable carriers and their formulations are known to those skilled in the art, and are described, for example, in Remington's Pharmaceutical Sciences (18th edition), ed. A. Gennaro, 1990, Mack Publishing Company, Easton, Pa.

The term “substantially identical” is used herein to describe a polypeptide or nucleic acid molecule exhibiting at least 50%, preferably at least 85%, more preferably at least 90%, and most preferably at least 95% identity to a reference amino acid or nucleic acid sequence. For polypeptides, the length of comparison sequences is at least 8 amino acids, preferably at least 16 amino acids, more preferably at least 25 amino acids, and most preferably 35 amino acids. For nucleic acid molecules, the length of comparison sequences is at least 24 nucleotides, preferably at least 50 nucleotides, more preferably at least 75, nucleotides, and most preferably at least 110 nucleotides. Sequence identity is typically measured using sequence analysis software with the default parameters specified therein (e.g., Sequence Analysis Software Package of the Genetics Computer Group, University of Wisconsin Biotechnology Center, 1710, University Avenue, Madison, Wis. 53705). The polypeptides, peptides, and nucleic acid molecules of the invention can be identical or substantially identical to naturally occurring molecules, and thus may or may not include non-wild type sequences.

By “substantially pure peptide” or “substantially pure polypeptide” is meant a peptide, polypeptide, or a fragment thereof, which has been separated from the components that naturally accompany it. Typically, the peptide or polypeptide is substantially pure when it is at least 60%, by weight, free from the proteins and naturally occurring organic molecules with which it is naturally associated. A substantially pure peptide or polypeptide can be obtained, for example, by extraction from a natural source (e.g., an activated Treg cell), by expression of a recombinant nucleic acid molecule encoding the protein, or by chemically synthesizing the peptide or polypeptide. Purity can be measured by any appropriate method, e.g., by column chromatography, polyacrylamide gel electrophoresis, HPLC analysis, etc.

A protein is substantially free of naturally associated components when it is separated from those contaminants that accompany it in its natural state. Thus, a protein that is chemically synthesized or produced in a cellular system different from the cell from which it naturally originates is substantially free from its naturally associated components. Accordingly, substantially pure peptides and poly-peptides not only include those derived from eukaryotic organisms, but also those synthesized in E. coli or other prokaryotes. By “substantially pure DNA” or “isolated DNA” is meant DNA that is free of the genes that, in the naturally occurring genome of the organism from which the DNA is derived, flank the gene. The term thus includes, for example, a recombinant DNA that is incorporated into a vector; an autonomously replicating plasmid or virus; or the genomic DNA of a prokaryote or eukaryote; or DNA that exists as a separate molecule (e.g., a cDNA, or a genomic or cDNA fragment produced by PCR or restriction endonuclease digestion) independent of other sequences. It also includes a recombinant DNA that is part of a hybrid gene encoding additional polypeptide sequence.

By “transformation,” “transfection,” or “transduction” is meant any method for introducing foreign molecules into a cell. Lipofection, DEAE-dextran mediated transfection, microinjection, protoplast fusion, calcium phosphate precipitation, transduction (e.g., bacteriophage, adenoviral retroviral, lentiviral or other viral delivery), electroporation, and biolistic transformation are just a few of the methods known to those skilled in the art that can be used in the invention.

By “transformed cell,” “transfected cell,” or “transduced cell,” is meant a cell (or a descendent of a cell) into which a nucleic acid molecule (e.g., a DNA or RNA molecule) encoding a polypeptide of the invention has been introduced by means of recombinant DNA techniques.

By “promoter” is meant a minimal sequence sufficient to direct transcription. Promoter elements that are sufficient to render promoter-dependent gene expression controllable for cell type-specific, tissue-specific, temporal-specific, or inducible by external signals or agents can also be used in the invention; such elements can be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “expression vector” is meant a genetically engineered plasmid or virus, de-rived from, for example, a bacteriophage, adenovirus, retrovirus, lentivirus, pox-virus, herpesvirus, or artificial chromosome, that is used to transfer a peptide or polypeptide coding sequence, operably linked to a promoter, into a host cell, such that the encoded peptide or polypeptide is expressed within the host cell.

The term “isolated” with regard to a population of cells as used herein refers to a cell population which either has no naturally-occurring counterpart or has been separated or purified from other components, including other cell types, which naturally accompany it, e.g., in normal or diseased tissues such as lung, kidney, or placenta, tumor tissue such as colon cancer tissue, or body fluids such as blood, serum, or urine. Typically, an isolated cell population is at least two-fold, four-fold, or eight-fold enriched for a specified cell type when compared to the natural source from which the population was obtained.

The term “test compound” or “candidate molecule” or “candidate agent” or “modulator” or grammatical equivalents as used herein describes any molecule, either naturally occurring or synthetic, e.g., protein, polypeptide, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds an be optionally linked to a fusion partner, e.g., targeting compounds, rescue compounds, dimerization compounds, stabilizing compounds, addressable compounds, and other functional moieties.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

### DETAILED DESCRIPTION OF THE INVENTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As detailed herein, a set of genes has been identified whose expression is specifically upregulated in activated Treg cells as compared to resting Tregs (listed in Table 1). An additional set of genes has been identified whose expression is specifically downregulated in activated Treg cells as compared to resting Tregs (listed in Table 2). Aspects of the present invention, which are based on these findings, are described below.

Identifying and Isolating Activated Regulatory T cells (Tregs)

Aspects of the present invention include methods for identifying (or detecting) activated immunosuppressive regulatory T cells (Tregs) in a sample. By “immunosuppressive regulatory T cells” or “regulatory T cells” or “Tregs” (or equivalents thereof) is meant T cell subpopulations with immunosuppressive properties. Exemplary Tregs include naturally occurring CD4⁺CD25^{high} Treg cells, induced Treg cells, e.g. Tr1 and TH3 cells, as well as adaptive CD4⁺CD25^{high} Treg cells developing in the periphery by conversion of CD4⁺CD25⁻ T cells. Exemplary additional markers of Tregs include FOXP3, which has increased expression levels in Tregs, and CD127, which exhibits negative to low expression levels in Tregs. Activated Tregs as used herein refers to Tregs that have been contacted to one or more T cell activating agents. Any convenient T cell activating agent may be used and includes activating in in vivo or in vitro environments. Exemplary activating agents include, but are not limited to: cytokines (e.g., IL-2, IL-15, and the like), antibodies (e.g., anti-CD3, anti-CD28, and the like), antigens/peptides (e.g., as presented by an antigen presenting cell, present on an allogeneic cell, or a purified protein component, e.g., peptide-MHC multimer), cells, etc.

In certain embodiments, the method comprises assaying one or more Tregs in a sample to obtain a gene expression result and identifying the Tregs as activated Tregs based on the gene expression result. In certain embodiments, the gene expression result includes a gene expression result for one or more genes listed in Table 1 or Table 2, as shown below.

TABLE I
genes upregulated in activated Tregs

| | OMIM Ref. # | |
|---|---|---|
| | (other reference # | |
| | listed if OMIM # | |
| Probeset | not available) | Description |
| GI_19923194-S | *109770 | Homo sapiens carcinoembryonic antigen-related cell adhesion |
| | | molecule 1 (biliary glycoprotein) (CEACAM1), mRNA. |
| GI_21396476-A | *600147 | Homo sapiens mesenchyme homeo box 1 (MEOX1), transcript |
| | | variant 2, mRNA. |
| GI_4505898-S | *176795 | Homo sapiens pro-melanin-concentrating hormone (PMCH), |
| | | mRNA. |
| GI_4504982-S | *153619 | Homo sapiens lectin, galactoside-binding, soluble, 3 (galectin 3) |
| | | (LGALS3), mRNA. |
| GI_34222185-S | *611640 | Homo sapiens fibronectin type 3 and ankyrin repeat domains 1 |
| | | (FANK1), mRNA. |
| GI_31982942-S | *300292 | Homo sapiens forkhead box P3 (FOXP3), mRNA. |
| GI_21389504-S | *611195 | Homo sapiens multiple coiled-coil GABABR1-binding protein |
| | | (MARLIN1), mRNA (also called JAKMIP1). |
| GI_31542938-S | *601688 | Homo sapiens hydroxyprostaglandin dehydrogenase 15-(NAD) |
| | | (HPGD), mRNA. |
| GI_27894331-S | *147810 | Homo sapiens interleukin 1 receptor, type I (IL1R1), mRNA. |
| GI_26190613-S | (NCBI Reference | Homo sapiens rhotekin 2 (RTKN2), mRNA. |
| | Sequence: | |
| | NP_660350) | |
| GI_5032092-S | *109190 | Homo sapiens solute carrier family 1 (neutral amino acid |
| | | transporter), member 5 (SLC1A5), mRNA. |
| GI_5031706-S | *137207 | Homo sapiens glycoprotein A repetitions predominant (GARP), |
| | | mRNA. |
| GI_21361211-S | *123890 | Homo sapiens cytotoxic T-lymphocyte-associated protein 4 |
| | | (CTLA4), mRNA. |
| GI_42659320-S | (NCBI Reference | Homo sapiens hypothetical protein LOC170371 (LOC170371), |
| | Sequence: | mRNA. |
| | NP_001010863.1) | |
| GI_23238193-A | *603905 | Homo sapiens tumor necrosis factor receptor superfamily, |
| | | member 18 (TNFRSF18), transcript variant 2, mRNA. |
| GI_27894333-A | *147811 | Homo sapiens interleukin 1 receptor, type II (IL1R2), transcript |
| | | variant 2, mRNA. |
| GI_21361446-S | *600323 | Homo sapiens regulator of G-protein signalling 1 (RGS1), |
| | | mRNA. |
| GI_23312365-S | *191191 | Homo sapiens tumor necrosis factor receptor superfamily, |
| | | member 1B (TNFRSF1B), mRNA. |
| GI_4507232-S | *603597 | Homo sapiens suppressor of cytokine signaling 1 (SOCS1), |
| | | mRNA. |
| GI_33636753-S | *606239 | Homo sapiens zinc finger protein, subfamily 1A, 4 (Eos) |
| | | (ZNFN1A4), mRNA. |
| GI_18641378-S | *142860 | Homo sapiens major histocompatibility complex, class II, DR |
| | | alpha (HLA-DRA), mRNA. |

TABLE 2
genes downregulated in activated Tregs

| | OMIM Ref. # (other | |
|---|---|---|
| | reference # listed if | |
| | OMIM # not | |
| Probeset | available) | Description |
| GI_6031196-S | 173610 | Homo sapiens selectin P (granule membrane protein 140 kDa, |
| | | antigen CD62) (SELP), mRNA. |
| GI_40788016-S | *609461 | Homo sapiens phosphoprotein regulated by mitogenic pathways |
| | | (C8FW; TRIB1), mRNA. |
| GI_5453765-S | *602320 | Homo sapiens NEL-like 2 (chicken) (NELL2), mRNA. |
| GI_37059785-S | *609793 | Homo sapiens leucine rich repeat neuronal 3 (LRRN3; LERN3; |
| | | LRRN6C), mRNA. |
| GI_22538813-S | *187011 | Homo sapiens chemokine (C-C motif) ligand 5 (CCL5), mRNA. |
| GI_45007001-S | (NCBI Reference | LAG1 homolog, ceramide synthase 6 |
| | Sequence: | |
| | NP_982288) | |
| GI_4504234-S | *600784 | Homo sapiens granzyme K (serine protease, granzyme 3; tryptase |
| | | II) (GZMK), mRNA. |
| GI_4503680-S | | Homo sapiens Fc fragment of IgG binding protein (FCGBP), |
| | | mRNA. |
| GI_28610150-S | *146661 | Homo sapiens interleukin 7 receptor (IL7R), mRNA. |
| GI_32967598-A | *600465 | Homo sapiens ankyrin 3, node of Ranvier (ankyrin G) (ANK3), |
| | | transcript variant 2, mRNA. |
| GI_13375927-S | (GenBank: | Homo sapiens hypothetical protein FLJ11795 (FLJ11795), |
| | AK021857.1) | mRNA. |
| GI_28416955-S | (NCBI Reference | GTPase, IMAP family member 8 |
| | Sequence: | |
| | NP_783161) | |
| GI_27498268-S | (GenBank: | Homo sapiens hypothetical protein FLJ40584 (FLJ40584), |
| | AK097903.1) | mRNA. |
| GI_6996012-S | *140050 | Homo sapiens granzyme A (granzyme 1, cytotoxic T-lymphocyte- |
| | | associated serine esterase 3) (GZMA), mRNA. |
| GI_28372502-S | *608246 | Homo sapiens keratin protein K6irs (K6IRS2; KERATIN 72; |
| | | KRT72), mRNA. |
| GI_5803016-S | *164772 | Homo sapiens FBJ murine osteosarcoma viral oncogene homolog |
| | | B, mRNA |
| GI_6552332-S | *164810 | Homo sapiens FBJ murine osteosarcoma viral oncogene homolog, |
| | | mRNA |
| GI_9910205-S | (NCBI Reference | Homo sapiens chromosome 14 open reading frame 132 |
| | Sequence: | (C14orf132), mRNA. |
| | NR_023938.1) | |
| GI_35038527-S | *607505 | Homo sapiens PAS domain containing serine/threonine kinase |
| | | (PASK), mRNA. |
| GI_28416951-S | *608084 | Homo sapiens immunity associated protein 1 (IMAP1), mRNA. |
| GI_22507400-S | *607305 | Homo sapiens myelodysplastic syndrome 2 (MDS2), mRNA. |
| GI_28416948-S | *608086 | Homo sapiens immune associated nucleotide 4 like 1 (mouse) |
| | | (IAN4L1), mRNA. |
| GI_4502100-S | *151690 | Homo sapiens annexin A1 (ANXA1), mRNA. |
| GI_29029598-S | *300201 | Homo sapiens cysteinyl leukotriene receptor 1 (CYSLTR1), |
| | | mRNA. |
| GI_28416430-S | *608085 | Homo sapiens immunity associated protein 2 (HIMAP2), mRNA. |

The sample containing, or thought to contain, activated Tregs can be derived from any suitable source. Sample sources include, but are not limited to: blood, cord blood, bone marrow, and derivatives thereof; tissues, e.g., spleen, thymus, liver, kidney, skin, etc.; biopsy samples (such as those from a transplanted tissue or organ); cells cultured or derived in vitro, e.g., from resting T cells (e.g., resting regulatory T cells), progenitor cells, etc. This can be from healthy donors but also patients with specific diseases, e.g. but not limited to cancer patients or patients with autoimmune diseases.

In certain embodiments, a suitable initial source for the sample is a blood sample. The blood-derived sample may be derived from whole blood or a fraction thereof, where in certain embodiments the sample is derived from blood cells harvested from whole blood. Of particular interest as a sample source are peripheral blood mononuclear cells/lymphocytes (PBMCs/PBLs). Any convenient protocol for obtaining such samples may be employed, where suitable protocols are well known in the art (e.g., density gradient fractionation of a whole blood sample).

In practicing the subject methods, the sample is assayed to obtain an expression level evaluation (or gene expression result), e.g., expression profile, for one or more genes selected from Tables 1 and/or Table 2, where the term expression profile is used broadly to include a genomic expression profile, e.g., an expression profile of nucleic acid transcripts, e.g., mRNAs, of the one or more genes of interest, or a proteomic expression profile, e.g., an expression profile of one or more different proteins, where the proteins/polypeptides are expression products of the one or more genes of interest. As such, in certain embodiments the expression level of only one gene in Table 1 or Table 2 is evaluated. In yet other embodiments, the expression level of two or more genes from Table 1 and/or Table 2 is evaluated, e.g., 3, 4 or all genes in Table 1 and/or Table 2. In certain embodiments, the expression level of one or more additional gene other than those listed in Tables 1 and Table 2 is also evaluated. It is noted here that an expression profile that includes an evaluation of the expression level of any combination of genes in Tables 1 and 2 finds use in identifying Tregs in a sample, including evaluating the expression of all genes listed in Tables 1 and Table 2.

In the broadest sense, the expression evaluation may be qualitative or quantitative. As such, where detection is qualitative, the methods provide a reading or evaluation, e.g., assessment, of whether or not the target analyte, e.g., nucleic acid or protein, is present in the cells being assayed (or screened). In yet other embodiments, the methods provide a quantitative detection of whether the target analyte is present in the cells being assayed, i.e., an evaluation or assessment of the actual amount or relative abundance of the target analyte, e.g., nucleic acid and or protein, in the cells being assayed. In such embodiments, the quantitative detection may be absolute or, if the method is a method of detecting two or more different analytes, e.g., target nucleic acids in a sample, relative. As such, the term “quantifying” when used in the context of quantifying a target analyte, e.g., nucleic acid(s), in a sample can refer to absolute or to relative quantification. Absolute quantification may be accomplished by inclusion of known concentration(s) of one or more control analytes and referencing the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other. In addition, a relative quantitation may be ascertained using a control, or reference, sample as is commonly done in array based assays as well as in quantitative PCR/RT-PCR analyses (described in further detail below).

As noted above, genes/proteins that find use identifying activated Tregs, i.e., genes/proteins that are differentially expressed or present at different levels in activated Tregs, are shown ion Tables 1 and 2. Note that for the genes in these tables, detailed information, including precise sequence information, can be determined through the NCBI Entrez Gene database located at the website http(colon)//www(dot)ncbi.nlm.nih(dot)gov. The detailed information for each gene is then obtained by selecting “Gene” and searching for the GeneID No. listed in these tables.

In addition, other array assay function related genes may be evaluated, e.g., for assessing sample quality (3′- to 5′-bias in probe location), sampling error in biopsy-based studies, cell surface markers, and normalizing genes for calibrating hybridization results (exemplary genes in these categories can be found in U.S. patent application Ser. No. 11/375,681, filed on Mar. 3, 2006, which is incorporated by reference herein in its entirety).

In certain embodiments, the expression profile obtained is a genomic or nucleic acid expression profile, where the amount or level of one or more nucleic acids in the sample is determined, e.g., the nucleic acid transcript of the gene of interest. In these embodiments, the sample that is assayed to generate the expression profile employed is one that is a nucleic acid sample. The nucleic acid sample includes a plurality or population of distinct nucleic acids that includes the expression information of the phenotype determinative genes of interest of the cell or tissue being screened for activated Tregs. The nucleic acid may include RNA or DNA nucleic acids, e.g., mRNA, cRNA, cDNA etc., so long as the sample retains the expression information of the cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. In certain embodiments, the sample is prepared from a cell or tissue harvested from a subject, or patient, e.g., a blood sample or biopsy of tissue, using standard protocols, where cell types or tissues from which such nucleic acids may be generated include any tissue in which activated Tregs are potentially present, including, but not limited to, peripheral blood lymphocyte cells, etc., as reviewed above.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. In certain embodiments, such applications are hybridization assays in which a nucleic acid array that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the levels of one or more nucleic acids in a sample may be employed, including quantitative PCR, real-time quantitative PCR, and the like. (For general details concerning real-time PCR see Real-Time PCR: An Essential Guide, K. Edwards et al., eds., Horizon Bioscience, Norwich, U.K. (2004)).

Where the expression profile is a protein expression profile, any convenient protein quantitation protocol may be employed, where the levels of one or more proteins in the assayed sample is determined. Representative methods include, but are not limited to: proteomic arrays, flow cytometry, standard immunoassays (e g , immunohistochemistry, immunofluorescence, ELISA assays, western blots, immunoprecipitation, affinity chromatography, etc.), protein activity assays, including multiplex protein activity assays, etc. Following obtainment of the expression data, or expression profile, from the sample being assayed, the expression profile is analyzed. In certain embodiments, identification of activated Treg cells employs antibodies specific for a protein expressed from the genes listed in Tables 1 and/or 2, e.g., with monoclonal or polyclonal antibodies (unlabeled or directly conjugated) specific for the protein. Antibodies can be generated, for example, by immunization of mammalians, for example mouse, rat, goat, donkey, rabbit, etc., with polypeptides or peptides derived from the protein of interest with the addition of specific adjuvants or identified by phage display.

In certain embodiments, analysis includes comparing the expression profile with a reference or control profile to identify activated Tregs. The terms “reference” and “control” as used herein mean a standardized pattern of gene expression or levels of expression of certain genes to be used to interpret the expression signature (or gene expression result) of a sample. The reference or control profile may be a profile that is obtained from a cell/tissue known to have the desired phenotype, e.g., a sample containing activated Tregs, and therefore may be a positive reference or control profile. In addition, the reference/control profile may be from a cell/tissue known to not have the desired phenotype, e.g., a sample lacking activated Tregs (e.g., a sample containing non-activated Tregs), and therefore be a negative reference/control profile.

In certain embodiments, the obtained expression profile is compared to a single reference/control profile to obtain information regarding whether activated Tregs are present in the sample. In yet other embodiments, the obtained expression profile is compared to two or more different reference/control profiles. For example, the obtained expression profile may be compared to a positive and negative reference profile to obtain confirmed information regarding whether the sample contains activated Tregs, and, in certain embodiments, the relative or absolute numbers of activated Tregs present.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. The comparison step results in information regarding how similar or dissimilar the obtained expression profile is to the control/reference profile(s).

Also provided are databases of expression profiles for samples having known composition of activated Tregs. Such databases will typically comprise expression profiles of specific samples (e.g., cells, tissues, biopsies, etc.) that have a known activated Treg composition, e.g., expression profiles for samples that are positive or negative for the presence of activated Tregs.

The expression profiles and databases thereof may be provided in a variety of media to facilitate their use (e.g., in a user-accessible/readable format). “Media” refers to a manufacture that contains the expression profile information of the present invention. The databases of the present invention can be recorded on computer readable media, e.g. any medium that can be read and accessed directly by a user employing a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. Thus, the subject expression profile databases are accessible by a user, i.e., the database files are saved in a user-readable format (e.g., a computer readable format, where a user controls the computer). As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention, e.g., to and from a user. One format for an output means ranks expression profiles possessing varying degrees of similarity to a reference expression profile. Such presentation provides a skilled artisan (or user) with a ranking of similarities and identifies the degree of similarity contained in the test expression profile to one or more references profile(s).

Aspects of the invention include isolating activated immunosuppressive regulatory T-cells (Tregs) from a sample. In certain embodiments, activated Tregs are isolated from a sample according to the expression level of one or more genes listed in Table 1 and/or Table 2, e.g., using the activated Treg identification methods described in detailed above. Assessment of the expression level of the one or more genes in Tables 1 and/or 2 may be achieved in any convenient manner. For example, gene expression can be based on nucleic acid levels (e.g., mRNA), protein levels, readout of transcriptional activity of a gene (e.g., determining the level of a reporter gene under the control of the endogenous or a recombinant promoter/enhancer of a gene in Tables 1 or 2), or a combination thereof. Isolation of activated Tregs can be achieved using any convenient method, and include the isolation of viable and/or non-viable activated Tregs.

As discussed above, a sample from which activated Tregs are to be isolated (or a sample containing activated Tregs) can be derived from any suitable source. Sample sources include, but are not limited to: blood, cord blood, bone marrow, and derivatives thereof; tissues, e.g., spleen, thymus, liver, kidney, skin, etc.; biopsy samples (such as those from a transplanted tissue or organ); cells cultured or derived in vitro, e.g., from resting T cells (e.g., resting regulatory T cells), progenitor cells, etc.

In certain embodiments, a suitable initial source for the sample is a blood sample. The blood-derived sample may be derived from whole blood or a fraction thereof, where in certain embodiments the sample is derived from blood cells harvested from whole blood. Of particular interest as a sample source are peripheral blood mononuclear cells/lymphocytes (PBMCs/PBLs). Any convenient protocol for obtaining such samples may be employed, where suitable protocols are well known in the art (e.g., density gradient fractionation of a whole blood sample).

As indicated, isolation of activated Tregs from a sample can be achieved using any convenient method, where the activated Tregs are isolated based on the assessed expression level of one or more genes from Table 1 and/or 2. As such, no limitation in this regard is intended. Exemplary isolation methods include, but are not limited to: flow cytometry, affinity-based methods (e.g., panning), viability screening (e.g., using antibiotic resistance genes placed under the control of endogenous or recombinant promoter/enhancer elements of a gene in Table 1 or 2); visual identification and isolation (e.g., isolating cells under light or fluorescence microscopy), etc.

In certain embodiments, the activated Tregs are isolated by flow cytometry. For example, a cell sample containing activated Tregs can be contacted to one or more binding elements specific for proteins for the genes listed in Table 1 and/or 2 and isolated based on their binding element binding characteristics by fluorescence activated cell sorting (FACS) (e.g., the cells are bound by binding elements specific for one or more proteins from the genes in Table 1 and/or are not bound by binding elements specific for one or more proteins from the genes in Table 2). By “binding element” is meant any agent that binds preferentially to a target molecule (e.g., a target protein or antigen) under specific binding conditions. Exemplary binding elements are antibodies (and target specific-binding fragments thereof) as is well known in the art. In much of the discussion herein, the term antibody is used generically to refer to binding moieties. However, this is not meant to limit the scope of binding elements that find use in practicing aspects of the subject invention.

FACS is a well known method for isolating cells having specified characteristics, e.g., having a specific protein expression profile for one or more proteins. In certain embodiments, the protein(s) being assayed for is a cell surface expressed protein. In certain other embodiments, the protein(s) being assayed for is an intracellular protein. In certain of the latter embodiments, the cells being assayed can be fixed and/or permeabilized to allow entry of the antibody of interest (or other protein-specific binding moiety) into the cell. Various fixatives are known in the art, including formaldehyde, paraformaldehyde, formaldehyde/acetone, methanol/acetone, etc. Formaldehyde used at a final concentration of about 1 to 2% has been found to be a good cross-linking fixative. Permeabilizing agents are also known in the art, and include mild detergents, such as Triton X-100, NP-40, saponin, etc.; methanol, and the like. In certain embodiments, both cell surface and intracellular protein expression is assessed to isolate activated Tregs. Flow cytometry may also be used to detect the expression of reporter genes in cells, e.g., for isolation purposes, such as a reporter gene that provides a readout for the level of expression of a genes listed in Tables 1 and/or 2, e.g., a reporter gene whose expression is driven by the expression of a gene in Tables 1 and/or 2.

It is noted here that the isolation of activated Tregs may additionally include the assessment of the expression of one or more genes not listed in Table 1 or 2, including those associated with conventional T cells and/or Tregs, e.g., CD4, CD8, CD3, CD25, etc.

In certain embodiments, the cells of a cell sample from which activated Tregs are to be isolated are enriched prior to the isolation step(s). For example, a blood cell sample may be enriched for mononuclear cells, or even T cells, before isolation staining and sorting by FACS. Positive and negative enrichment steps for this purpose are well known in the art.

Aspects of the present invention include methods for producing activated Tregs, where the activation can be performed in vivo or in vitro. Producing activated Tregs includes contacting a non-activated Treg (or resting Treg) with a regulatory T cell activating composition, where the contacting induces activation of the Tregs. Regulatory T cell activating compositions can include any of a number of components, including: antigens, cytokines, cells or cell fractions, T cell stimulatory agents and combinations thereof. Non-limiting examples of regulator T cell activating components include: alloantigen, autoantigen (including tissues, cells, cell fragments or debris, purified polypeptides or peptides, etc., e.g., in combination with antigen-presenting cells), IL-2, IL-15 antigen presenting cells, allogeneic cells, anti-CD3 antibody (or binding fragments thereof), anti-CD28 antibody (or binding fragments thereof), anti-CD2 antibody (or binding fragments thereof), B7 (or CD28 binding fragments thereof), Concanavalin A, superantigens, MHC polymers, lectins (such as PHA), auto-antigens, phorbol ester, calcium ionophor, etc.

In certain embodiments, activated Tregs are produced prior to identifying and/or isolating the activated Tregs (as detailed above).

### Isolated Activated Tregs and Pharmaceutical Compositions Thereof

In certain embodiments, the subject invention provides an isolated population of activated Tregs, e.g., isolated using the methods detailed above. In certain embodiments, the isolated activated Tregs have an increased expression of one or more genes listed in Table 1 (upregulated genes) and/or a decreased expression of one or more genes listed in Table 2 (downregulated genes) as compared to non-activated (or resting) Treg cells. Isolated populations of activated Tregs are enriched for activated Tregs as compared to the source from which they are derived (e.g., a blood sample or an in vitro culture). In certain embodiments, the isolated population is enriched by a factor of 2 or more, 5 or more, 10 or more, 20 or more 50 or more, 75 or more, 100 or more, 200 or more, 500 or more, 1000 or more, etc. Thus, isolated populations of cells enriched for activated Tregs can contain 2% or more, 5% or more, 10% or more, 25% or more, 50% or more, 75% or more, 90% or mroe, 95% or more, 99% or more, up to and including 100% activated Tregs. Isolated activated Treg compositions may include other cell types or T cell subpopulations, either present in the source from which the activated Tregs were isolated or cells added to the composition thereafter.

In certain embodiments, the isolated activated Tregs are antigen specific, e.g., an alloantigen, an autoantigen, etc. In certain of these embodiments, the antigen specificity of the activated Tregs may be assessed as part of the isolation process, e.g., by using MHC/peptide multimers (e.g., tetramers) for identification of the antigen-specificity of the T cell receptor of the activated Tregs. In certain other embodiments, the antigen specificity of the activated Treg (or its non-activated precursor) is determined prior to the isolation.

Isolated populations of activated Tregs find use in a number of research and therapeutic applications.

Aspects of the present invention include a pharmaceutical composition comprising isolated activated Tregs as described herein. Such pharmaceutical compositions find use, e.g., in suppressing or preventing an aberrant or pathological immune response in a subject (or patient), where in certain embodiments, the isolated activated Tregs are antigen specific (as described above).

In certain embodiments, pharmaceutical preparations of isolated activated Tregs described herein are administered to patients suffering from, for example, aberrant immune responses and/or autoimmune diseases, including allograft rejection or graft versus host disease.

In certain embodiments, pharmaceutical preparations of isolated activated Tregs described herein can be administered to patients using methods generally known in the art. Such methods include without limitation injecting or introducing the activated Tregs into a patient. In some embodiments, activated Tregs are introduced into a patient via intravenous administration. In further embodiments, additional reagents such as buffers, salts or other pharmaceutically acceptable additives may be administered in combination with activated Tregs.

After introducing the cells into the patient, the effect of the treatment may be evaluated using methods known in the art. Examples of such evaluations can include without limitation: measuring titers of total or of specific immunoglobulins, renal function tests, tissue damage evaluation, cellular analysis (e.g., the presence/absence of specific T cell subsets, including activated Tregs), and the like. Treatment using activated Tregs of the invention may be repeated as needed or required. For example, the treatment may be done once a week for a period of weeks, or multiple times a week for a period of time, for example 3-5 times over a two week period. Over time, the patient may experience a relapse of symptoms, at which point the treatments may be repeated.

In one exemplary aspect, the invention provides a method of treating an aberrant immune response or an autoimmune disease in a patient, including the step of administering activated regulatory T cells to the patient.

In one embodiment, the activated regulatory T cells administered to a patient are generated in vitro, e.g., using a T cell activator, including without limitation antigen (or antigen presenting cell), cells, IL-2, anti-CD3, anti-CD28, or any combination thereof.

The isolated activated regulatory T cells employed in the treatment of a subject may be syngeneic or allogeneic, i.e., derived from the recipient or from a donor, respectively.

The pharmaceutical composition or medicament may furthermore comprise pharmaceutically acceptable carriers known to a person skilled in the art. The pharmaceutical composition or medicament is preferably suitable for is suitable to treat diseases with enhanced immunity including, but not limited to, autoimmune diseases, graft versus host disease and graft rejections.

### Screening for Treg Activating or Inhibitory Agents

In certain embodiments, the present invention provides methods of identifying an agent (or candidate agent) as a modulator of immunosuppressive regulatory T cell activation. A “modulator” as used in this context includes an agent that promotes that activation of Tregs or an agent that inhibits the activation of Tregs. Such aspects of the present invention can be considered screening assays, e.g., methods for screening candidate agents for Treg activation modulatory activity.

In certain embodiments, the methods include contacting a sample containing one or more immunosuppressive regulatory T cell with a candidate agent (or agents) followed by analyzing the contacted immunosuppressive regulatory T cells for the expression of at least one gene in Table 1 or Table 2 to obtain a gene expression result. Based on the gene expression result, the candidate agent (or agents) can be identified as a modulator of immunosuppressive regulatory T cells.

In certain embodiments, the candidate agent is identified as an activator of immunosuppressive regulatory T cells when the expression of at least one gene in Table 1 is increased in the immunosuppressive regulatory T cells when contacted with (e.g., cultured in the presence of) the candidate agent, e.g., CEACAM1. In certain embodiments, the candidate agent is identified as an activator of immunosuppressive regulatory T cells when the expression of at least one gene in Table 2 is decreased in the immunosuppressive regulatory T cells when the cells are contacted with the candidate agent, e.g., SELP.

In certain embodiments, the candidate agent is identified as an inhibitor of immunosuppressive regulatory T cell activation when, in the presence of the candidate agent, the expression of at least one gene in Table 1 is not increased in the immunosuppressive regulatory T cells under activating conditions (e.g., contacted with a Treg activating agent(s); e.g., anti-CD3 and IL-2). In other words, a candidate agent is identified as a Treg activation inhibitor if it can block the increased expression of one or more genes in Table 1 when a Treg is placed under activation conditions. Likewise, a candidate agent is identified as a Treg activation inhibitor if it can block the decreased expression of one or more genes in Table 2 when a Treg is placed under activation conditions.

In embodiments of the screening assays detailed above, the expression level of a combination of genes from Tables 1 and/or 2 are analyzed, where in certain embodiments, two or more, five or more, 10 or more, 15 or more, 25 or more up to and including all of the genes in Tables 1 and/or 2 are analyzed for their expression levels. Further, additional genes not listed in Tables 1 or 2 can be analyzed in these assays. In certain embodiments, the expression of one or more control genes is assessed, where the one or more control genes can be expressed at a constant level during regulatory T cell activation (e.g., a housekeeping gene). In further embodiments, the additional genes may be employed to determine the relative number of regulatory T cells between different samples (e.g., in samples with and without the candidate agent; or “loading controls”).

### Isolated Proteins, Antibodies and Uses Therefore

Aspects of the invention include samples containing isolated proteins encoded by the genes listed in Tables 1 and 2 as well as peptides derived therefrom. The production and isolation of proteins and peptides can be achieved in any convenient manner and is often at the discretion of a user of the isolated protein/peptide.

A wide variety of expression systems can be used to recombinantly produce proteins/peptides (e.g., polypeptides, fragments, fusion proteins, and amino acid sequence variants, etc.). The proteins/peptides can be produced in prokaryotic hosts (e.g., E. coli) or in eukaryotic hosts (e.g., S. cerevisiae, insect cells, such as Sf9 cells, or mammalian cells, such as COS-1, NIH 3T3, Jurkat, 293, 293T, or HeLa cells). These cells are commercially available from, for example, the American Type Culture Collection, Rockville, Md. (also see, e.g., Ausubel et al., Curent Protocols in Molecular Biology, John Wiley & Sons, New York, N.Y., 1998). The method of transformation and the choice of expression vehicle (e.g., expression vector) depends on the host system selected. Transformation and transfection methods are described, e.g., by Ausubel et al., supra, and expression vehicles can be chosen from the numerous examples that are known in this field.

First, a nucleic acid molecule encoding the protein/peptide of interest is introduced into a plasmid or other vector, which is then used to transform living cells. Constructs in which a cDNA containing the entire coding sequence, a fragment of such coding sequence, an amino acid variations of such coding sequence, or fusion proteins, inserted in the correct orientation into an expression plasmid, can be used for protein expression. In certain embodiments, the expression is controlled by a constitutive promoter, whereas in certain other embodiments an inducible or tissue-specific promoter is used. Vectors may further include eukaryotic and/or prokaryotic “origin of replication” sequences, which allow for their autonomous replication within the host cell/organism; sequences that encode genetic traits that allow vector-containing cells to be selected in the presence of otherwise toxic drugs (such as antibiotics); and sequences that increase the efficiency with which the synthesized mRNA is translated. Stable, long-term vectors can be maintained as freely replicating entities within cells by using regulatory elements of, for example, viruses (e.g., the OriP sequences from the Epstein Barr Virus genome). Cell lines can also be produced that have the vector integrated into genomic DNA, and, in this manner, the gene product is produced on a continuous basis.

Once the expression vector is constructed, it is introduced into an appropriate host cell by transformation, transfection, or transduction techniques that are known in the art, including calcium chloride transformation, calcium phosphate transfection, DEAE-dextran transfection, electroporation, microinjection, protoplast fusion, and liposome-mediated transfection. The host cells that are transformed with the vectors of this invention can include (but are not limited to) E. coli or other bacteria, yeast, fungi, insect cells (using, for example, baculoviral vectors for expression), human, mouse, or other animal cells. Mammalian cells can also be used to express recombinant proteins using a vaccinia virus expression system, as is described by Ausubel et al., supra.

In vitro expression systems for producing a protein (or peptides, fusions, polypeptide fragments, or mutated versions thereof) may also be employed, e.g., using the T7 late promoter expression system. Plasmid vectors containing late promoters and the corresponding RNA polymerases from related bacteriophages such as T3, T5, and SP6 can also be used for in vitro production of proteins from cloned DNA. E. coli can also be used for expression using an M13 phage such as mGPI-2. Furthermore, vectors that contain phage lambda regulatory sequences, or vectors that direct the expression of fusion proteins, for example, a maltose binding protein fusion protein or a ‘glutathione s-transferase fusion protein, also can be used for expression in E. coli.

Eukaryotic expression systems permit appropriate post-translational modifications to expressed proteins. Transient transfection of a eukaryotic expression plasmid allows the transient production of proteins/peptides of interest by a transfected host cell. The proteins/peptides can also be produced by a stably-transfected mammalian cell lines. A number of vectors suitable for stable transfection of mammalian cells are available to the public (e.g., see Pouwels et al, Cloning Vectors: A Laboratory Manual, 1985, Supp. 2, 987), as are methods for constructing such cell lines (see, e.g., Ausubel et al., supra). In one example, cDNA encoding a protein (or peptide, protein, fragment, mutant, or fusion protein thereof) is cloned into an expression vector that includes the dihydrofolate reductase (DHFR) gene. Integration of the plasmid and, therefore, integration of the gene encoding the protein of interest into the host cell chromosome is selected by inclusion of 0.01-300 μM methotrexate in the cell culture medium (as is described by Ausubel et al., supra). This dominant selection can be accomplished in most cell types. Recombinant protein expression can be increased by DHFR-mediated amplification of the transfected gene. Methods for selecting cell lines bearing gene amplifications are described by Ausubel et al., supra. These methods generally involve extended culture in medium containing gradually increasing levels of methotrexate. The most commonly used DHFR-containing expression vectors are pCVSEII-DHFR and pAdD26SV(A) (described by Ausubel et al., supra). The host cells described above or, preferably, a DHFR-deficient CHO cell line (e.g., CHO DHFR-cells, ATCC Accession No. CRL 9096) are among those most preferred for DHFR selection of a stably-transfected cell line or DHFR-mediated gene amplification. Other drug markers can be analogously used.

Another eukaryotic expression system that may be employed is the trc expression vector system using, for example, the vector pTrcHis, which is available from Invitrogen (Karlsruhe, Germany). If desired, this system can be used to express the proteins/peptides of interest fused to a protein tag, for example, the myc, His, or XPRESS tag as known in the art.

Once a recombinant protein/peptide is expressed, it can be isolated from the expressing cells by cell lysis followed by well known protein purification techniques, such as affinity chromatography. For example, a protein having an XPRESS tag can be purified using an anti-XPRESS antibody attached to a column by standard methods (see, e.g., Ausubel et al., supra). Once isolated, the recombinant protein can, if desired, be purified further, e.g., by high performance liquid chromatography (HPLC; e.g., see Fisher, Laboratory Techniques in Biochemistry and Molecular Biology, Work and Burdon, Eds., Elsevier, 1980).

Peptides (or proteins) can also be produced by chemical synthesis where desired (e.g., by the methods described in Solid Phase Peptide Synthesis, 2nd ed., 1984, The Pierce Chemical Co., Rockford, Ill., or by other methods known to those skilled in the art of peptide synthesis).

Aspects of the invention include antibodies specific for the proteins encoded by the genes listed in Tables 1 and 2. Antibodies can be prepared, for example, using the isolated proteins/peptides described above by methods well-established in the art including, but not limited to: administration of an antigen presenting cell (APC) presenting a peptide derived from the protein of interest (e.g., APCs pulsed with a peptide or engineered to express a protein/peptide that is processed and presented by the APC) to a host; immunization of a host with a protein/peptide of interest, in the presence or absence of an adjuvant(s); delivering a nucleic acid molecule encoding a protein of interest to a cell (or host) so that it can be processed (e.g., by an antigen presenting cell) in the host to produce antibodies or using a protein of interest to bind antibodies (or antigen binding fragments thereof) expressed by a phage library. Isolated peptides for use in generating antibodies (or cytotoxic cells, as detailed below) can be specifically selected to bind to major histocompatibility complex molecules.

In certain embodiments, antibody production the protein of interest or a MHC-binding peptide thereof is administered to an animal in association with an adjuvant. For example, a chemical antigen (e.g., Freund's incomplete adjuvant; cytoxan; an aluminum compound, such as aluminum hydroxide, aluminium phosphate, or aluminium hydroxyphosphate; liposomes; ISCOMS; microspheres; protein chochleates; vesicles consisting of nonionic surfactants; cationic amphiphilic dispersions in water; oil/water emulsions; muramidyldipeptide (MDP) and its de-rivatives such as glucosyl murarnid dipeptide (GMDP), threonyl-MDP, murametide and murapalmitin; and QuilA and its subfractions; as well as various other compounds such as monophosphoryl-lipid A (MIPLA); gamma-inulin; calcitriol; and loxoribine) can be used.

A biological response modifier, which is a soluble mediator that affects induction of an immune response, can also be used as an adjuvant. For example, cytokines (e.g., IL2 and GM-CSF), chemokines, co-stimulatory molecules (e.g., B7, ICAM, class I monoclonal antibodies, stem cell factor, and stimulated T cells) can be used. Also, bacterial products, such as toxins or, preferably, subunits or fragments thereof that have reduced (if any) toxicity, but maintained adjuvant activity. Additional types of adjuvant molecules that can be used in the invention include, for example, biological modifiers of the death response (e.g., apoptosis sensitizers) and compounds or treatment that increases the susceptibility of the target cell to treatment, such as radiation and chemotherapy.

Antibodies specific for the proteins of Tables 1 and 2 find us in numerous diagnostic, research and therapeutic applications.

For example, antibodies specific for the proteins of interest can be used to identify and/or isolate activated Tregs as detailed above (e.g., using flow cytometry). In certain of these applications, the antibodies may be labeled (e.g., with fluorescent or enzymatic labels).

Antibodies specific for a protein of interest can further be administered to a subject/patient (e.g., a monoclonal or polyclonal antibody, humanized, chimeric or non-humanized) that modulates that activity of the activated Treg, either positively or negatively. For example, an antibody specific for a protein encoded by a gene listed in Table 1 (the genes having increased expression in activated Tregs) may induce the death of an activated Treg in vivo.

Aspects of the invention further include administering to the patient cytotoxic T lymphocytes (CTL) (autologous or allogeneic) that eliminate Treg cells in a protein specific, major histocompatibility complex-restricted fashion (i.e., by targeting activated Tregs based on their expression of one or more gene in Table 1). The CTL can be generated, for example, by activation with antigen presenting cells displaying a protein/peptide of interest in the context of a major histocompatibility complex molecule. The invention also includes an alternative method of treating a patient to eliminate activated Treg cells that express a Table—gene encoded protein. This method involves administering to the patient an antigen presenting cell (APC) that activates in the patient a cytotoxic T lymphocyte that kills the cell in a protein specific, major histocompatibility complex-restricted fashion. The APC can be engineered to present the protein/peptide of interest (i.e., encoded by a gene in Table 1) in the context of a major histocompatibility complex molecule.

Activated Treg cells can further be reduced/eliminated in a patient by administering to the patient an isolated/purified protein (described above), e.g., in association with an adjuvant, whereby the patient mounts an immune reaction targeting activated Tregs expressing the protein of interest. As an alternative, a nucleic acid molecule encoding the protein of interested (e.g., an expression vector) can be administered to a subject/patient. The nucleic acid molecule is expressed in the patient so that it can be processed by an antigen presenting cell in the patient, which activates a cytotoxic T lymphocyte in the patient to induce cell death of the cell that expresses the protein of interest.

Each of the methods described above can also include treatment based around a second (or more) Treg cell associated antigen or a peptide thereof that binds to MHC. In any of the methods described above, the patient can have Treg cells that express a protein of interest. APCs used in these methods can be, for example, a dendritic cell or a CD40-activated B cell. The peptide employed in these methods can bind to a class I or a class II major histocompatibility complex (MHC) molecule.

Conventional pharmaceutical practice can be employed to provide suitable formulations or compositions to administer to a subject. As such, pharmaceutical compositions can be administered within a pharmaceutically-acceptable diluent, carrier, or excipient, in unit dosage form. Administration can begin before a patient is symptomatic. Any appropriate route of administration can be employed, for example, administration can be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations can be in the form of liquid solutions or suspensions; for oral administration, formulations can be in the form of tablets or capsules; and for intraanasal formulations, in the form of powders, nasal drops, or aerosols. An adjuvant, e.g., as listed above, can be included with the formulation.

Methods well known in the art for making formulations are found, for example, in Remington's Pharmaceutical Sciences, (18th edition), ed. A. Gennaro, 1990, Mack Publishing Company, Easton, Pa. Formulations for parenteral administration can, for example, contain excipients, sterile water, or saline, polyalkylene glycols such as polyethylene glycol, oils of vegetable origin, or hydrogenated napthalenes. Biocompatible, biodegradable lactide polymer, lactidelglycolide co-polymer, or polyoxyethylene-polyoxypropylene copolymers can be used to control the release of the compounds. Other potentially useful parenteral delivery systems for the above mentioned peptides, proteins/polypeptides, and nucleic acid molecules include ethylene-vinyl acetate copolymer particles, osmotic pumps, implantable infusion systems, and liposomes. Formulations for inhalation can contain excipients, for example, lactose, or can be aqueous solutions containing, for example, polyoxyethylene-9-lauxyl ether, glycocholate and deoxy-cholate, or can be oily solutions for administration in the form of nasal drops, or as a gel.

Aspects of the invention also includes a method of assessing a sample from a subject/patient for cytotoxic T lymphocytes specific for a protein encoded by a gene listed in Table 1. The sample can be obtained from the patient before, during, or after a treatment is administered to the patient. A sample can also be obtained, for example, before and after treatment.

Aspects of the invention include an ex vivo generated cytotoxic T lymphocyte that specifically kills an activated Treg that expresses, or has an increased expression of, a protein encoded by a gene listed in Table 1.

Aspects of the invention include pharmaceutical compositions or medicaments containing one or more protein encoded by one or more genes listed in Table 1 for use in vaccination on a subject. These include, without limitation, full length proteins, MHC-binding fragments of the proteins, as well as fusion proteins. Peptides or polypeptides can include 8, 9, 10, 11, 12, or more amino acid stretches having sequence identity with a region of the protein of interest (i.e., listed in Table 1). For example, the peptides can include nine amino acid stretches, in which seven, eight, or all nine of the amino acids in the protein of the invention are identical to a region of nine amino acids (or more) in the protein of interest, including up to the full-length of the protein. In certain embodiments, the polypeptides can contain additional amino acid stretches that do not correspond to the amino acid sequence of the proteins encoded by the genes listed in Table 1.

Aspects of the invention include employing antibodies against one or more of the proteins of interest in diagnostic assays that measure the presence of activated Treg cells in a test sample. For example, the presence (or increased levels) of proteins encoded by the genes listed in Table 1 (or negative or decreased levels of proteins encoded by the genes listed in Table 2) in a Treg cell indicates that the Treg is an activated Treg. The sample can be from any suitable source, e.g., from a subject who has received a therapy or a transplant. The Treg sample may be enriched for a specific subset of cells, e.g., Tregs having a TCR that is specific for an antigen of interest (e.g., sorted using peptide/MHC multimers). The diagnostic assay may be compared to a control or reference sample, e.g., Treg cells from a pre-therapy sample from the subject. Results from the diagnostic assay may be used to determine efficacy of treatment and/or to devise a subsequent therapeutic regimen.

Purified peptides of the protein of the invention can also be useful for diagnostic assays. For example, proteins can be used to measure the presence of specific antibodies or CTLs in a test sample. For example, the presence (or increased levels) of CTLs and/or antibodies specific for a protein of interest (e.g., a protien encoded by a gene listed in Table 1) in a sample from a vaccinated subject/host, relative to a control or reference sample (such as a pre-vaccination sample from the patient), indicates that the subject/host has mounted a protein specific immune response.

As is mentioned above, in addition to the vaccination methods described above, which result in the activation of antigen-specific, MHC-restricted CTLs in vivo, such cells (i.e., antigen-specific, MHC-restricted CTLs) can be generated in vitro, and then administered to patients. Any cell that expresses an endogenous or exogenously-introduced major histocompatibility antigen-encoding gene can be used to present a peptide of the proteins of the invention (e.g., encoded by the genes listed in Table 1) to generate CTLs in vitro. In one variation of this approach, a peptide-presenting cell expresses an endogenously or exogenously-introduced gene coding for a protein of interest. In another variation, the antigen presenting cells are pulsed with the proteins/peptides (e.g., MHC-binding peptides), and the pulsed cells are then used to generate CTLs, e.g., for administration to a patient. The CTLs used in these methods are obtained from the patient to whom they are to ultimately be administered (i.e., the cells are autologous). Alternatively, donor cells (i.e., allogeneic cells) can be used in this method. Finally, methods in which any of the above-described immunotherapeutic approaches are combined are included in the invention.

Aspects of the invention include in vivo expression of genes of interest, e.g., those listed in Table 1. Retroviral, adenoviral, lentiviral, poxviral, and other viral vectors are suited as nucleic acid expression vectors for in vivo delivery, because they show efficient infection and/or integration and expression; (Cayouette and Gravel, Hum. Gene Ther.; 8: 423-430 (1997); Kido et al., Curr. Eye Res.; 15: 833-844 (1996); Miyoshi et al., Proc. Natl. Acad. Sci. USA; 94: 10319-10323 (1997); Naldini et al., Science; 272: 263-267 (1996)). For example, any DNA fragment that encodes a protein or peptide of interest can be cloned into a retroviral or lentiviral vector and transcribed via its endogenous promoter, via an exogenous promoter, via a promoter specific for the target cell type of interest, or, in the case of retroviral or lentiviral vectors, via the viral long terminal repeat. Other viral vectors that can be used include adenovirus, adenoassociated virus, poxviruses, such as vaccinia virus or bovine papilloma virus, or a herpes virus, such as Epstein-Barr Virus.

Gene transfer in vivo can also be achieved by non-viral means. For example, a plasmid vector of interest can be injected directly into skeletal muscle or cardiac muscle by previously described methods (Wolff et al., Science; 247: 1465-1468 (1990)). Expression vectors injected into skeletal muscle in situ are taken up into muscle cell nuclei and used as templates for expression of their encoded proteins. Gene transfer into cells within the tissues of a living animal also can be achieved by Lipofection (Brigham et al., Am. J. Med. Sci.; 298: 278-281 (1989); Felgner et al., Proc. Natl. Acad. Sci. USA; 84: 7413-7417 (1987); Ono et al., Neurosci. Lett.; 117: 259-263 (1990)), or asialoorosomucoid-polylysine conjugation (Wu et al., J. Biol. Chem.; 264: 16985-16987 (1989); Wu and Wu, J. Biol. Chem.; 263: 14621-14624 (1988)), and analogous methods.

Retroviral vectors, adenoviral vectors, adenovirus-associated viral vectors, or other viral vectors also can be used to deliver genes encoding proteins/peptides of interest in cells ex vivo. Numerous vectors useful for this purpose are generally known (Anderson, Science; 224: 340 (1984); Cornetta et al., Prog. Nucleic Acid. Res. Mol. Biol.; 36: 311-322 (1989); Eglitis et al., Adv. Exp. Med. Biol; 241: 19-27 (1988); Friedmann, Science; 244: 1275-1281 (1989); Johnson, Chest; 107: 77S-83S (1995); Le Gal La Salle et al., Science; 259: 988-990 (1993); Miller, Hum. Gene. Ther.; 1: 5-14 (1990); Moen, Blood Cells; 17: 407-416 (1991); Tolstoshev and Anderson, Curr. Opin. Biotechnol.; 1: 55-61 (1990)). Retroviral vectors are particularly well developed and have been used in clinical settings (Rosenberg et al., N. Engl. J. Med.; 323: 570-578 (1990)); Anderson et al., U.S. Pat. No. 5,399,346).

Gene transfer into cells ex vivo can also be achieved by delivery of non-viral vectors, such as expression plasmids, using methods such as calcium phosphate or DEAE dextran transfection, electroporation, and protoplast fusion. Liposomes can also be potentially beneficial for delivery of DNA into a cell.

Cells that are to be transduced or transfected ex vivo can be obtained from an animal or a patient (e.g., peripheral blood cells, such as Treg cells, B cells or dendritic cells, bone marrow stem cells, or cells from a tumor biopsy) prior to transfection, and reintroduced after transfection. However, the cells also can be derived from a source other than the patient or animal undergoing gene transfer.

In the constructs described above, protein expression can be directed from any suitable promoter (e.g., the human cytomegalovirus (CMV), simian virus 40 (SV40), EF1-α, or metallothionein promoters), and regulated by any appropriate mammalian regulatory element. For example, if desired, enhancers known to preferentially direct gene expression in skeletal muscle cells can be used to direct protein expression for vaccination in situ. The enhancers used can include, without limitation, those that are characterized as tissue- or cells specific in their expression.

### Systems and Kits

Also provided are reagents, systems and kits thereof for practicing one or more of the above-described methods. As such, the subject reagents, systems and kits thereof may vary greatly.

In certain embodiments, reagents of interest include reagents specifically designed for use in identifying and/or purifying activated Tregs from a sample. The term system refers to a collection of reagents, however compiled, e.g., by purchasing the collection of reagents from the same or different sources. The term kit refers to a collection of reagents provided, e.g., sold, together.

One type of such reagent is an array of probe nucleic acids in which the activated Treg specific genes of interest are represented (e.g., the genes listed in Tables 1 and/or 2). A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

In certain embodiments, the arrays include probes for at least 1 of the genes listed in Table 1 and/or Table 2. As such, probes for any combination of genes in Table 1 and/or Table 2 may be employed. Therefore, in certain embodiments, the number of genes that are from Table 1 that are represented on the array is at least 2, at least 3, at least 4, at least 5, at least 8 or more, including all of the genes listed in Table 1 and/or Table 2. The subject arrays may include only those genes that are listed in Table 1 and/or Table 2 or they may include additional genes that are not listed in Table 1 and/or Table 2, such as probes for genes whose expression pattern can be used to evaluate additional sample characteristics, e.g., for assessing sample quality (3′- to 5′-bias in probe location), sampling error, other cell surface markers, and normalizing genes for calibrating hybridization results; and the like.

In certain embodiments, systems and kits may include a collection of gene specific primers that is designed to selectively amplify genes of interest form a sample containing, or suspected of containing, activated Tregs (e.g., using a PCR-based technique, e.g., real-time RT-PCR). Gene specific primers and methods for using the same are described in U.S. Patent No. 5,994,076, the disclosure of which is herein incorporated by reference. Of particular interest are collections of gene specific primers that have primers for at least 1 of the genes listed in one Table 1 and/or Table 2, often a plurality of these genes, e.g., at least 2, 4, 8 or more. In certain embodiments, all of genes that are from Table 1 and/or Table 2 have primers in the collection. The subject gene specific primer collections may include only those genes that are listed in Table 1 and/or Table 2, or they may include primers for additional genes that are not listed in Table 1 and/or Table 2, such as probes for genes whose expression pattern can be used to evaluate additional characteristics, e.g., for assessing sample quality (3′- to 5′-bias in probe location), sampling error in biopsy-based studies, cell surface markers, and normalizing genes for calibrating hybridization results; and the like.

The systems and kits of the subject invention may include the above-described arrays and/or gene specific primer collections. The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

The subject systems and kits may also include an activated Treg determination element, which element is, in many embodiments, a reference or control expression profile that can be employed, e.g., by a suitable computing means, to aid in the determination of the number of activated Tregs in a sample. Representative phenotype determination elements include databases of expression profiles, e.g., reference or control profiles, as described above.

In certain embodiments, systems and kits of the invention include reagents for isolating activate Tregs from a sample, including antibodies specific for one or more proteins encoded by the genes listed in Tables 1 and/or 2. In certain embodiments, the antibodies are labeled, e.g., detectably labeled, such that the binding of the antibodies to cells can be detected, e.g., by flow cytometry. Antibodies specific for proteins other than those encoded by the genes in Tables 1 and 2 can also be present, including those for cell surface markers for T cells (including Tregs), e.g., CD3, CD4, CD25, CD127, etc. Antibodies and other reagents for negative, as well as positive, selection may also be present.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

Patients and clinical parameters: For isolation of CD4⁺ CD25^{high} Treg cells and conventional CD4⁺ CD25⁻ T cells following approval by the institutional review board of the University of Cologne, Germany, peripheral blood from 4 healthy individuals and 4 patients with chronic lymphatic leukemia (CLL) was obtained after informed consent. Patients included for phenotypical or functional analysis were untreated prior to investigation. Staging was performed according to the Binet classification for CLL.

For isolation of CD4⁺ CD127^{low} CD25⁺ FOXP3⁺ Treg cells and CD4⁺ CD127⁺ CD25⁻ low/int FOXP3⁻ T cells peripheral blood from 4 healthy individuals was obtained after informed consent and approval by the institutional review board.

Isolation of PBMC from healthy donors and CLL patients: Peripheral blood mono-nuclear cells (PBMC) were obtained using Ficoll/Hypaque (Amersham, Uppsala, Sweden) density centrifugation. Therefore heparinized blood samples were diluted 1:1 with RPMI and layered onto 15 ml of Ficoll/Hypaque. After centrifugation for 30 min at 450 g, the interphase was collected, washed twice with RPMI and cryoperserved in the gas phase of a liquid nitrogen tank in 10% DMSO and 90% FCS until further processing.

Isolation of CD4⁺ CD25^{high} Treg cells and conventional CD4⁺ CD25⁻ T cells: Briefly, after washing twice with RPMI, CD4 MACS® Beads (Miltenyi Biotec) were used for the isolation of CD4⁺ T cells from PBMC according to the manufacturer's recommendations. After staining with CD25-PE and CD4-APC (both from BD) according to the manufacturer's recommendations, CD4⁺ C25⁻ and CD4⁺ CD25^{high} T cells were purified using a FACSDiVa™ Cell Sorter (BD Biosciences). Purity of the CD4⁺ CD25^{high} Treg cell population was routinely checked and resulted in >90% CD4⁺ CD25^{high} Treg cells after purification. After isolation cells were either lysed directly in TRIzol reagent (Invitrogen Life Technologies) and stored at −80° C. until further processing or lysed after an additional stimulated for 20 hours.

Polyclonal stimulation CD4⁺ CD25^{high} Treg cells and conventional CD4⁺ CD25⁻ T cells with CD3 and interleukin-2: To assess the effect of short-term polyclonal stimulation on the gene expression of CD4⁺ CD25^{high} Treg cells and conventional CD4⁺ CD25⁻ T cells, 1×10⁶ cells of the respective T cell population were activated in X-Vivo 15 (BioWhittaker) with anti-CD3 (0.5 μg/ml, OKT-3) and IL-2 (10 IU/ml, Proleukin, Chiron) for 20 hours.

RNA preparation, microarray hybridization and microarray data processing: RNA isolation and quantification was performed according to the manufacturer's recommendations (Illumina). Biotin labeled cRNA preparation was performed using the Ambion Illumina RNA amplification kit (Ambion Europe, Huntington, Cambridgeshire, UK) according to the manufacturer's recommendations. 1.5 μg biotin labeled cRNA was hybridized to Sentrix whole genome bead chips 6×2 (Illumina, San Diego, Calif., USA) according to the manufacturer's recommendations and scanned on the Illumina BeadStation 500×. For data collection, assessment and statistical analysis we used Illumina BeadStudio software and dCHIP 1.3. The following filtering criteria were used for selection of differentially expressed genes: fold change ≧2, absolute difference in signal intensity between group means ≧50 and p value ≦0.05 (paired t-test).

Isolation of CD4⁺ CD127^{low} CD25⁺ Treg cells, conventional CD4⁺ CD127⁺ CD25^{low} and activated CD4⁺ CD 127^{low} CD25^{int} T cells, RNA preparation, microarray hybridization and microarray data processing: According to our analysis as well as two reports published in 2006 CD127, the IL-7 receptor α-chain, is the most specific surface molecule down regulated in CD4⁺ CD25⁺ Treg cells. The expression of CD127 is inverse correlated with the expression of the Treg cell specific transcription factor FOXP. We therefore wanted to confirm the expression of the identified differentially expressed genes in the CD4⁺ CD127^{low} CD25⁺ Treg cell population as this population should be further enriched in Treg cells than the CD4⁺ CD25⁺ Treg cell population.

After thawing CD4⁺ T cells were purified using the BD IMag™ Cell Separation System (BD Biosciences) with CD4 Particles according to the manufacturer's recommendations. After staining with CD127-Alexa 647, CD25-PE and CD4-PerCP-Cy5.5 (both from BD) according to the manufacturer's recommendations, CD4⁺ CD127^{low} CD25⁺ Treg cells, conventional CD4⁺ CD 127⁺ CD25^{low} and activated CD4⁺ CD 127⁺ CD25^{int} T cells were purified using a FACSDiVa™ Cell Sorter (BD Biosciences). Purity of the CD4⁺ CD127^{low} CD25⁺ FOXP3⁺ Treg cell population as measured by FOXP3 Alexa 488 staining was routinely checked and resulted in >95% CD4⁺ CD25^{high} Treg cells after purification. RNA isolation and quantification, Biotin labeled cRNA preparation, hybridization, scanning, and statistical analysis was performed as described before. The criteria used for selection of differentially expressed genes were defined as already mentioned.

### Example 1

### Identification of Genes in Activated Tregs

The aim of this study was to identify Genes showing unique expression in activated regulatory T cells (Tregs) compared to unstimulated Treg cells were identified as follows and were designated the “core transcriptome” of Tregs. A total of 192 individual experiments interrogating conventional and Tregs in different states of activation were performed. CD4⁺ T cells were isolated from peripheral blood and were subsequently separated into CD25⁺ and CD25⁻ cells. CD25⁻ cells were either left unstimulated (resting) or were exposed to different stimuli. These included activation through CD3 and CD28 antibodies with or without addition of inhibitory signals (TGFβ, CTLA4, PGE2, PD1 and IL-10). CD25⁺ cells were either left unstimulated, were exposed to activation by IL-2 or were expanded using Rapamycin. All samples are present in replicates with least n=3.

To identify potential candidate genes the following filtering criteria were used for selection of differentially expressed genes: fold change ≧2, absolute difference in signal intensity between group means ≧50 and p value ≦0.05 (paired t-test). Genes adhering to these filtering criteria were then selected as genes showing unique expression in activated regulatory T cells.

Genes satisfying these filtering criteria were identified as the major discriminating genes between activated and resting Treg cells (Table 1 and 2).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An isolated population of cells comprising at least 50% activated immunosuppressive regulatory T cells, wherein said activated immunosuppressive regulatory T cells express at least one gene selected from Table 1.

2. The isolated population of cells according to claim 1, wherein said activated immunosuppressive regulatory T cells are antigen specific.

3. The isolated population according to claim 2, wherein said antigen is selected from: an alloantigen, a tumor antigen and an autoantigen.

4. The isolated population according to claim 1, wherein said activated immunosuppressive regulatory T cells are low or negative for the expression of at least one gene in Table 2.

5. The isolated population according to claim 1, wherein said activated immunosuppressive regulatory T cells are derived from peripheral blood mononuclear cells (PBMC).

6. A method of identifying activated immunosuppressive regulatory T cells in a sample comprising: screening T cells in said sample for the expression of at least one gene in Table 1 or Table 2; and identifying said detected T cells as activated immunosuppressive regulatory T-cells based on said screening.

7. The method according to claim 6, wherein said method further comprises contacting said sample to a regulatory T cell activator composition prior to said screening step.

8. The method according to claim 7, wherein said regulatory T cell activator composition comprises one or more of: antigen, cytokines, cells and T cell stimulatory agents.

9. The method according to claim 8, wherein said antigen is selected from: a tumor antigen, an alloantigen and an autoantigen.

10. The method according to claim 8, wherein said cytokine is selected from: IL-2 and IL-15.

11. The method according to claim 8, wherein said cells are selected from: tumor cells, antigen presenting cells and allogeneic cells.

12. The method according to claim 8, wherein said T cell stimulatory agents are selected from: anti-CD3 antibody, anti-CD28 antibody, anti-CD2 antibody, Concanavalin A and superantigens.

13. The method according to claim 6, wherein said method further comprises isolating said identified activated immunosuppressive regulatory T-cells.

14. The method according to claim 6, wherein said sample is a peripheral blood mononuclear cell (PBMC) sample.

15. A method of identifying an agent as modulator of immunosuppressive regulatory T cells comprising:
contacting a sample comprising immunosuppressive regulatory T cells with a candidate agent;
screening the immunosuppressive regulatory T cells for the expression of at least one gene in Table 1 or Table 2; and
identifying said candidate agent as a modulator of immunosuppressive regulatory T cells based on said screening.

16. The method of claim 15, wherein said candidate agent is identified as an activator of immunosuppressive regulatory T cells when the expression of at least one gene in Table 1 is increased in said immunosuppressive regulatory T cells.

17. The method of claim 15, wherein said contacting step further comprises contacting said sample with an immunosuppressive regulatory T cell activating agent, wherein said candidate agent is identified as an inhibitor of immunosuppressive regulatory T cell activation when the expression of at least one gene in Table 1 is not increased in said immunosuppressive regulatory T cells.

18. A pharmaceutical composition for suppressing a pathological immune response in a subject, wherein said pharmaceutical composition comprises isolated activated immunosuppressive CD4⁺ CD25⁺ regulatory T cells.

19. The pharmaceutical composition according to claim 18, wherein said isolated activated immunosuppressive CD4⁺ CD25⁺ regulatory T cells are antigen specific.

20. The pharmaceutical composition according to claim 19, wherein said antigen is selected from: an alloantigen and an autoantigen.

21. The pharmaceutical composition according to claim 18, wherein said isolated activated immunosuppressive CD4⁺ CD25⁺ regulatory T cells are derived from said subject.

22. The pharmaceutical composition according to claim 18, wherein said isolated activated immunosuppressive CD4⁺ CD25⁺ regulatory T cells are derived from a donor.
